(19) United States
(12) Patent Application Publication
(10) Pub. No.: US 2013/0337565 A1
(43) Pub. Date: Dec. 19, 2013

(54) Method and Apparatus for Patterning Cells

(71) Applicant: MedTrain Technologies, LLC, Hillsborough, NC (US)

(72) Inventors: Albert J. Banes, Hillsborough, NC (US); Colin Patrick Frazier, Raleigh, NC (US); Chris James Wimmer, Raleigh, NC (US)

(21) Appl. No.: 13/921,887

(22) Filed: Jun. 19, 2013

Related U.S. Application Data
(60) Provisional application No. 61/661,631, filed on Jun. 19, 2012.

(51) Int. Cl. C12M 1/42

(52) U.S. Cl. CPC C12M 35/04
USPC 435/401; 435/289.1

(57) ABSTRACT

The present invention is directed to tissue engineering and, more particularly, to devices and methods that are used to pattern or deposit cells to simulate a tissue type in two dimensions or in three dimensions or a cell migration device, a materials testing device for cell proliferation, migration or cell seeder for the Bioflex® flexible bottom culture plates or comparable culture plates. The present invention operates by providing negative pressure to create multiple troughs or indentations for cells to attach and grow on or in the Bioflex® flexible bottom culture plates. The present invention is also directed to a device and method for simulating a tissue wound using the above devices.

### CROSS REFERENCE TO RELATED APPLICATION

This application is related to and claims the benefit of U.S. Provisional Patent Application 61/661,631 filed on Jun. 19, 2012, which is herein incorporated by reference in its entirety.

### BACKGROUND OF THE INVENTION

Connective tissue cells from muscle, bone, tendon, ligament, and cartilage respond to mechanical loading. Many types of devices have been developed to apply static strain to cells. These devices include weights placed upon cells grown on a distensible membrane, and a forcing frame in which cells on a distensible substrate are statically stretched.

A particular device, conceived by the present inventor, applies static tension or compression to cultured cells grown on a deformable substrate. The deformation of the substrate is regulated by pressure controlled by a solenoid valve and a timer. In one embodiment of the device, a vacuum is used to downwardly deform a polystyrene surface on which tendon cells are attached. The cells respond by altering their synthesis of cytoskeletal proteins. This device in one embodiment is a computer controlled device that provides regimens of strain having defined duration, frequency, and amplitude. A culture plate that allows easy growth of cells on a flexible bottom culture plate is used with this device. For reference, see U.S. Pat. Nos. 7,738,682; 6,998,265; 6,472,202; 6,218,178; 6,048,723; 6,037,141; 5,518,909; 4,839,280 and U.S. Pat. Publication No. 2007/0077653, which are herein incorporated by reference in their entirety.

### SUMMARY OF THE INVENTION

The present invention is directed to tissue engineering and, more particularly, to devices and methods that are used to pattern or deposit cells to simulate a tissue type in two dimensions or in three dimensions or a cell migration device, a materials testing device for cell proliferation, migration or cell seeder for the Bioflex® flexible bottom culture plates, provided by Flexcell International Corporation, or comparable culture plates. The present invention is also directed to a device for simulating a tissue wound using the above devices.

In one embodiment the present invention is a cell culture apparatus including a post comprising a recessed area bounded by a continuous perimeter, a flexible membrane overlaying at least part of the post and overlaying the recessed area, and a plurality of holes within the recessed area configured to communicate a vacuum to draw said flexible membrane into the recessed area bounded by the continuous perimeter. In another embodiment the recessed area of the post is of a predetermined shape selected from a group consisting of a circular shape, a rectangular shape, a shape of a tricuspid heart valve, a bifurcated shape, a cusp shape, an elongate trough shape, and a shape of a plurality of arms connected to each other via a connecting portion. In another embodiment, the cell culture apparatus includes a ring comprising a body having a first end and a second end, and a cylindrical sidewall extending between the first end and the second end, said cylindrical sidewall defining a cavity, said first end of said ring being configured to fit inside the recessed area of the post reducing the length of the continuous perimeter configured to contact the flexible membrane. In a further embodiment said first end of said ring defines at least one channel configured to cooperate with said plurality of holes to apply vacuum to draw said flexible membrane to conform into the recessed area bounded by the continuous perimeter of the post. In another embodiment the recessed area forms a circular shape. In another embodiment the recessed area forms a rectangular shape. In another embodiment the recessed area forms a shape of a tricuspid heart valve. In another embodiment the recessed area forms a rectangular shape with a bifurcation at one side of the rectangular shape. In another embodiment the recessed area forms a bifurcated shape. In another embodiment the recessed area forms a cusp shape. In another embodiment the recessed area forms an elongate trough shape. In another embodiment the recessed area is shaped as a plurality of arms connected to each other via a connecting portion.

In another embodiment the present invention is a cell culture apparatus including a post comprising a recessed area bounded by a continuous perimeter, a flexible membrane overlaying at least part of the post and overlaying the recessed area, a ring comprising a body having a first end and a second end, and a cylindrical sidewall extending between the first end and the second end, said cylindrical sidewall defining a cavity, said second end of said ring being configured to fit inside the recessed area of the post reducing the length of the continuous perimeter configured to contact the flexible membrane, and a plurality of holes within the recessed area configured to communicate a vacuum to draw said flexible membrane into the recessed area bounded by the continuous perimeter. In another embodiment said ring is readily removable and is readily installable into the recessed area of the post. In another embodiment said second end of said ring defines at least one channel configured to cooperate with said plurality of holes to apply vacuum to draw said flexible membrane to conform into the cavity defined by the cylindrical sidewall of said ring. In another embodiment the recessed area of the post is of a predetermined shape selected from a group consisting of a circular shape, a rectangular shape, a shape of a tricuspid heart valve, a bifurcated shape, a cusp shape, an elongate trough shape, and a shape of a plurality of arms connected to each other via a connecting portion. In another embodiment the recessed area forms a circular shape. In another embodiment the recessed area forms a rectangular shape. In another embodiment the recessed area forms a shape of a tricuspid heart valve. In another embodiment the recessed area forms a rectangular shape with a bifurcation at one side of the rectangular shape. In another embodiment the recessed area forms a bifurcated shape. In another embodiment the recessed area forms a cusp shape. In another embodiment the recessed area forms an elongate trough shape. In another embodiment the recessed area is shaped as a plurality of arms connected to each other via a connecting portion.

In another embodiment the present invention is a method of patterning cells, including the steps of placing a flexible membrane over a recessed area of a predetermined shape bounded by a continuous perimeter of a post, drawing the flexible membrane into the recessed area of the post, depositing at least one cell and a gel onto the flexible membrane in the recessed area, allowing the gel to polymerize, and allowing the cells to proliferate. In another embodiment the method further includes the step of positioning a flexible cell anchor on the flexible membrane so that at least a portion of the flexible cell anchor is positioned in the recessed area. In another embodiment the flexible cell anchor is constructed from a non-woven mesh material. In another embodiment the step of drawing the flexible membrane into the recessed area of the post comprises communicating a vacuum to the flexible membrane. In another embodiment the method further includes the step of inserting a ring into the recessed area reducing the length of the continuous perimeter, wherein said ring comprising a body having a first end and a second end, and a cylindrical sidewall extending between the first end and the second end, said cylindrical sidewall defining a cavity, said second end of said ring being configured to fit inside the recessed area. In another embodiment said ring is readily removable and is readily installable into the recessed area of the post. In another embodiment the recessed area of the post is of a predetermined shape selected from a group consisting of a circular shape, a rectangular shape, a shape of a tricuspid heart valve, a bifurcated shape, a cusp shape, an elongate trough shape, and a shape of a plurality of arms connected to each other via a connecting portion. In another embodiment the recessed area forms a circular shape. In another embodiment the recessed area forms a rectangular shape. In another embodiment the recessed area forms a shape of a tricuspid heart valve. In another embodiment the recessed area forms a rectangular shape with a bifurcation at one side of the rectangular shape. In another embodiment the recessed area forms a bifurcated shape. In another embodiment the recessed area forms a cusp shape. In another embodiment the recessed area forms an elongate trough shape. In another embodiment the recessed area is shaped as a plurality of arms connected to each other via a connecting portion.

In another embodiment the present invention is a cell culture apparatus including a post comprising a recessed area bounded by a continuous perimeter, a flexible membrane overlaying at least part of the post and overlaying the recessed area, and a plurality of holes within the recessed area configured to communicate a vacuum to draw said flexible membrane into the recessed area bounded by the continuous perimeter, wherein the recessed area of the post is of a predetermined shape selected from a group consisting of a rectangular shape, a tricuspid heart valve shape, a bifurcated shape, a cusp shape, an elongate trough shape, and a plurality of arms connected to each other via a connecting portion. In another embodiment the invention further includes a ring comprising a body having a first end and a second end, and a sidewall extending between the first end and the second end, said sidewall defining a cavity, said first end of said ring being configured to fit inside the recessed area of the post reducing the length of the continuous perimeter configured to contact the flexible membrane. In another embodiment said first end of said ring defines at least one channel configured to cooperate with said plurality of holes to apply vacuum to draw said flexible membrane to conform into the recessed area bounded by the continuous perimeter of the post. In another embodiment the predetermined shape is the rectangular shape. In another embodiment the predetermined shape is the tricuspid heart valve shape. In another embodiment the predetermined shape is the rectangular shape, wherein the rectangular shape has a bifurcation at one side of the rectangular shape. In still another embodiment the predetermined shape is the bifurcated shape. In another embodiment the predetermined shape is the cusp shape. In another embodiment the predetermined shape is the elongate trough shape. In another embodiment the predetermined shape is the plurality of arms connected to each other via a connecting portion.

In another embodiment the present invention is a cell culture apparatus including a recessed area bounded by a continuous perimeter, a flexible membrane overlaying at least part of the recessed area, a ring comprising a body having a first end and a second end, and a sidewall extending between the first end and the second end, said sidewall defining a cavity, said second end of said ring being configured to fit inside the recessed area, and a plurality of holes within the recessed area configured to communicate with a vacuum to draw said flexible membrane into the cavity defined by said sidewall when said second end of said ring is inside the recessed area. In another embodiment said second end of said ring defines at least one channel configured to cooperate with said plurality of holes to apply vacuum to draw said flexible membrane to conform into the cavity defined by said sidewall of said ring. In another embodiment the recessed area is of a predetermined shape selected from a group consisting of a circular shape, a rectangular shape, a tricuspid heart valve shape, a bifurcated shape, a cusp shape, an elongate trough shape, and a plurality of arms connected to each other via a connecting portion. In another embodiment the predetermined shape is the circular shape. In another embodiment the predetermined shape is the rectangular shape. In another embodiment the predetermined shape is the tricuspid heart valve shape. In another embodiment the predetermined shape is the rectangular shape, wherein the rectangular shape has a bifurcation at one side of the rectangular shape. In another embodiment the predetermined shape is the bifurcated shape. In another embodiment the predetermined shape is the cusp shape. In another embodiment the predetermined shape is the elongate trough shape. In another embodiment the predetermined shape is the plurality of arms connected to each other via a connecting portion. In another embodiment the cavity is of a predetermined shape selected from a group consisting of a circular shape, a rectangular shape, a tricuspid heart valve shape, a bifurcated shape, a cusp shape, an elongate trough shape, and a plurality of arms connected to each other via a connecting portion.

In another embodiment the present invention is a method of patterning cells, including the steps of providing a cell culture apparatus comprising, a flexible membrane over a recessed area having a predetermined shape bounded by a continuous perimeter, drawing the flexible membrane into the recessed area, depositing at least one cell and a gel onto the flexible membrane in the recessed area, allowing the gel to polymerize, and allowing the cells to proliferate, wherein the recessed area is of a predetermined shape selected from a group consisting of a rectangular shape, a tricuspid heart valve shape, a bifurcated shape, a cusp shape, an elongate trough shape, and a plurality of arms connected to each other via a connecting portion. In another embodiment the method further includes the step of positioning a flexible cell anchor on the flexible membrane so that at least a portion of the flexible cell anchor is positioned in the recessed area. In another embodiment the flexible cell anchor is constructed from a non-woven mesh material. In another embodiment the step of drawing the flexible membrane into the recessed area comprises communicating a vacuum to the flexible membrane. In another embodiment the predetermined shape is the rectangular shape. In another embodiment the predetermined shape is the tricuspid heart valve shape. In another embodiment the predetermined shape is the rectangular shape, wherein the rectangular shape has a bifurcation at one side of the rectangular shape. In another embodiment the predetermined shape is the bifurcated shape. In another embodiment the predetermined shape is the cusp shape. In another embodiment the predetermined shape is the elongate trough shape. In another embodiment the predetermined shape is the plurality of arms connected to each other via a connecting portion.

### BRIEF DESCRIPTION OF THE DRAWINGS

FIG. 1 shows a perspective of a 24-post post plate of an embodiment of the present invention;

FIG. 2 shows another perspective view of the 24-post post plate, shown in FIG. 1, of an embodiment of the present invention;

FIG. 3 shows a close up perspective view of the 24-post post plate, shown in FIG. 1, of an embodiment of the present invention;

FIG. 4 shows a plan view of the 24-post post plate, shown in FIG. 1, of an embodiment of the present invention;

FIG. 5 shows a close up perspective view of the 24-post post plate, shown in FIG. 1, coupled to a complementary culture plate of an embodiment of the present invention;

FIG. 6 shows a perspective view of the 24-post post plate, shown in FIG. 1, coupled to a complementary culture plate, shown in a sectional view along line A-A of FIG. 5 of an embodiment of the present invention;

FIG. 7 shows a perspective cross sectional view along line B-B of FIG. 5 of the 24-post post plate, shown in FIG. 1, coupled to a complementary culture plate of an embodiment of the present invention;

FIG. 8 shows cells stained with crystal violet that were grown for 48 hours on the membranes of a 24 well HTP plate with a flexible membrane growth surface;

FIG. 9 shows a close-up of the cells stained with crystal violet that were grown for 48 hours on the membranes of a 24 well HTP plate with a flexible membrane growth surface, shown in FIG. 8;

FIG. 10A shows a plan view of a 6-post post plate showing six different embodiments of cell pattern designs to seed cells in predetermined geometries;

FIG. 10B shows a perspective view of the 6-post post plate showing six different embodiments of cell pattern designs to seed cells in predetermined geometries, shown in FIG. 10A;

FIG. 11A shows a plan view of a 6-post post plate for seeding cells in rectangular or square patterns;

FIG. 11B shows a perspective view of the 6-post post plate for seeding cells in rectangular or square patterns, shown in FIG. 11A;

FIG. 12A shows a plan view of a rectangle with curved short ends loading post assembly;

FIG. 12B shows a perspective view of the rectangle with curved short ends loading post assembly, shown in FIG. 12 A;

FIG. 13 shows a 24 well HTP plate that has had 24 trapazoidal three dimensional cell-hydrogel constructs seeded in trapazoidal trough shaped recessed areas with construct ends attached to a nonwoven nylon mesh that is attached to the membrane at the poles (at anchor ends of the nylon) but attached to the cell-gel construct at the construct ends at anchor tabs in the nylon;

FIG. 14 shows a top perspective view of a 6-post post plate for a tricuspid valve shape cell pattern;

FIG. 15 shows a close up perspective view of a post having a recessed area for a tricuspid valve shape cell pattern;

FIG. 16 shows top plan view of a 6-post post plate for tricuspid valves;

FIG. 17 shows a bottom perspective view of a 6-post post plate for tricuspid valves;

FIG. 18 shows an exploded view of a tricuspid heart valve assembly where vacuum pressure is pulled from a vacuum pump through the baseplate;

FIG. 19 shows a perspective view of a tricuspid heart valve assembly where vacuum pressure is pulled from a vacuum pump through the baseplate;

FIG. 20 shows a top view of a tricuspid heart valve assembly where vacuum pressure is pulled from a vacuum pump through the baseplate;

FIG. 21 shows a top plan view of a 6-post post plate with a series of rings designed to reduce the exposed area of the flexible membrane;

FIG. 22 shows a bottom perspective view of the ring shown in FIG. 21;

FIG. 23 shows a cross sectional view along line C-C, shown in FIG. 11A, of the post with a flexible membrane over the post of FIG. 11A;

FIG. 24 shows a cross sectional view along line C-C, shown in FIG. 11A, of the post with the flexible membrane over the post of FIG. 11A when vacuum is communicated to the flexible membrane;

FIG. 25 shows a cross sectional view along line C-C, shown in FIG. 11A, of the post with the flexible membrane over the post of FIG. 11A when vacuum is communicated to the flexible membrane, with cells and cell medium being deposited on the flexible membrane;

FIG. 26 shows a perspective cross sectional view along line C-C, shown in FIG. 11A, of the post with the flexible membrane over the post of FIG. 11A when vacuum is communicated to the flexible membrane with the cell medium being changed on the flexible membrane;

FIG. 27 shows a perspective cross sectional view along line C-C, shown in FIG. 11A, of the post with the flexible membrane over the post of FIG. 11A when vacuum is communicated to the flexible membrane during an incubation period;

FIG. 28 shows a perspective view of the post with the flexible membrane over the post of FIG. 11A when vacuum is not communicated to the flexible membrane at the beginning of a cell migration assay;

FIG. 29 shows a diagram of the process of cell migration as it may occur in the cell migration assay shown in FIG. 28;

FIG. 30 shows perspective view of an embodiment of a post with a trough shaped recessed area coupled to a well to grow cell cultures according to the present invention;

FIG. 31 shows a plan view of a cell construct using the post with a trough shaped recessed area of FIG. 30;

FIG. 32 shows a cross sectional view along line D-D, shown in FIG. 31, of a post with a trough shaped recessed area coupled to a well to grow cell cultures of FIG. 30;

FIG. 33 shows a cross sectional view along line D-D, shown in FIG. 31, of a post with a trough shaped recessed area coupled to a well to grow cell cultures of FIG. 30, with vacuum being communicated to the flexible membrane and cells with medium being deposited on the flexible membrane;

FIG. 34 shows a cross sectional view along line D-D, shown in FIG. 31, of a post with a trough shaped recessed area coupled to a well to grow cell cultures of FIG. 30, with no vacuum being communicated to the flexible membrane with cells adhered to the flexible membrane, forming a cell construct;

FIG. 35 shows a perspective view of a post with a trough shaped recessed area coupled to a well to grow cell cultures of FIG. 30, with no vacuum being communicated to the flexible membrane with cells adhered to the flexible membrane, forming a cell construct as also shown in FIG. 34; and

FIG. 36 shows a perspective view of a post with a bifurcated shaped recessed area coupled to a well to grow cell cultures forming a cell construct in the recessed area.

### DETAILED DESCRIPTION OF THE INVENTION

A complete understanding of the invention will be obtained from the following description when taken in connection with the accompanying figures wherein like reference characters identify like parts throughout.

For purposes of the description hereinafter, the terms “upper”, “lower”, “right”, “left”, “vertical”, “horizontal”, “top”, “bottom”, and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

An object of the present invention is to cause cells to attach and grow in specific areas of a flexible-bottom culture well, such as the 6 well Bioflex® culture plate or the high throughput (HTP) 24 well culture plate. The initial use is to plate cells only over the recessed area of a post placed beneath each well membrane. This is particularly important for the smaller diameter wells in the 24 well plate (1.6 cm diameter wells) because the annular portion of each well becomes about 50% of the growth surface area of the well. If one applies strain to these cells without rejecting the peripheral cells from the population, those peripheral cells will be hyper-stretched while the ones over the loading post will be properly stretched.

Another object of the present invention is to control the geometric shape of an adherent cell population, or group of cells, at a given location in a culture well, which has other, more broad ramifications for the tissue engineering of medically relevant devices and implants. One use of this method is to produce tissue simulates such as a heart valve with multiple leaflets; a cornea, dermis or dermis in combination with epidermis to make a skin simulate. However, person skilled in the art will recognize additional uses and advantages of the present invention.

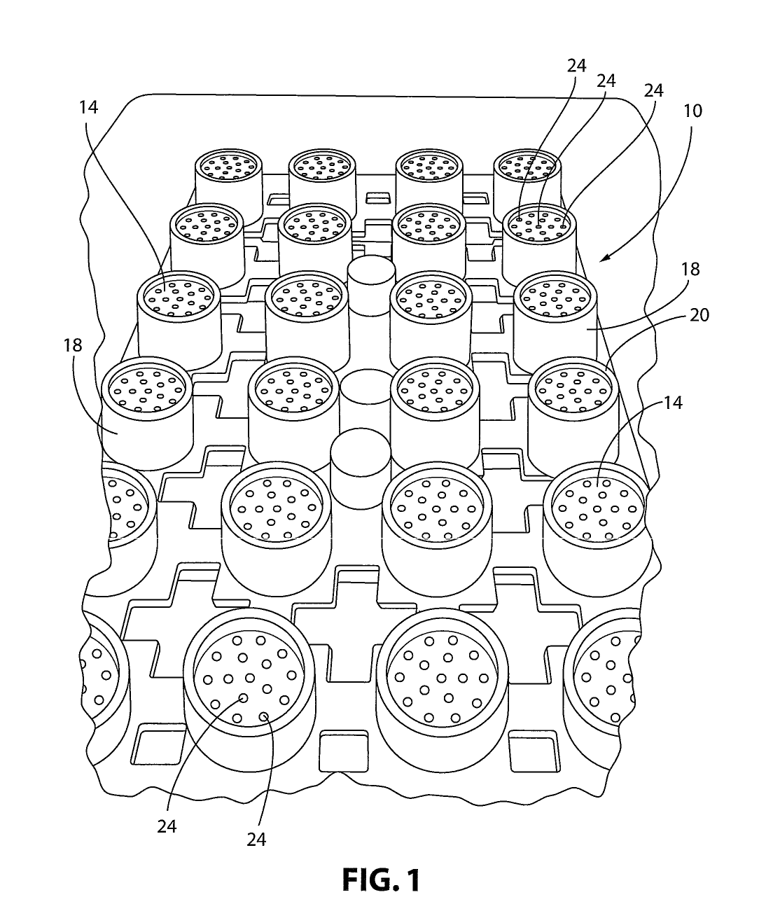

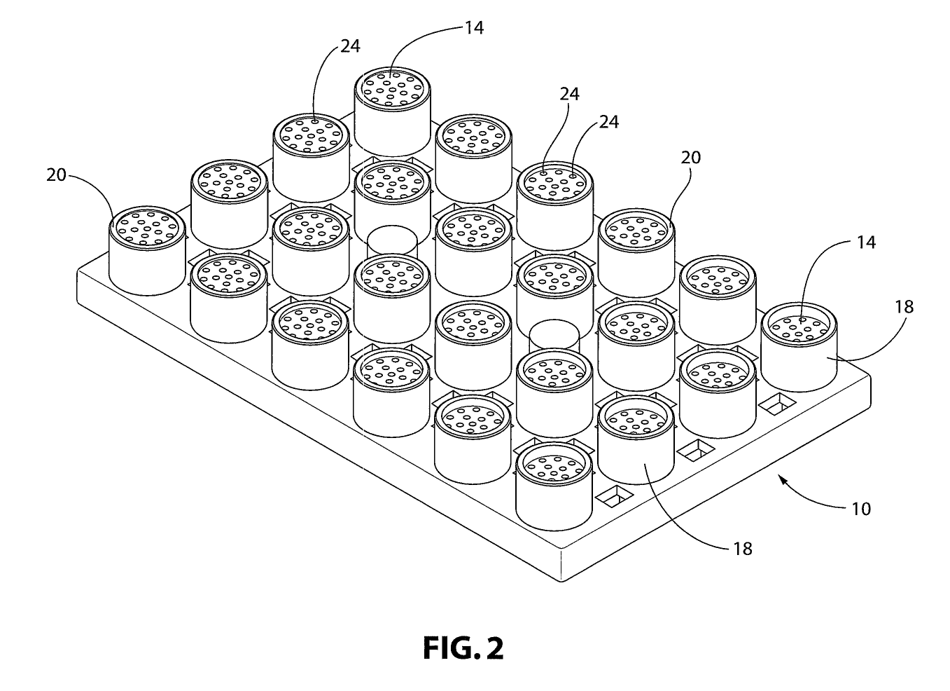

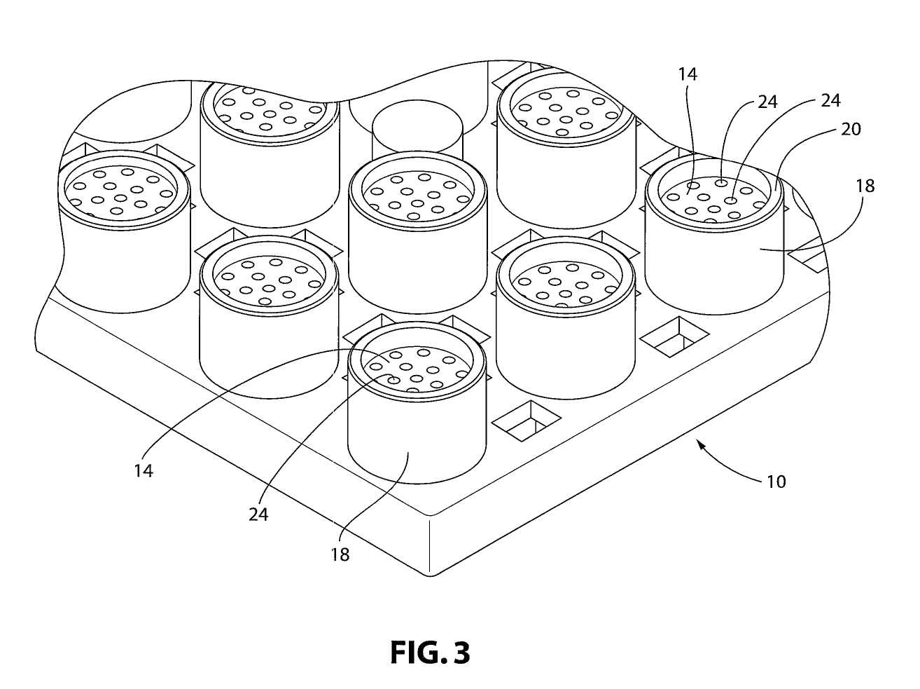

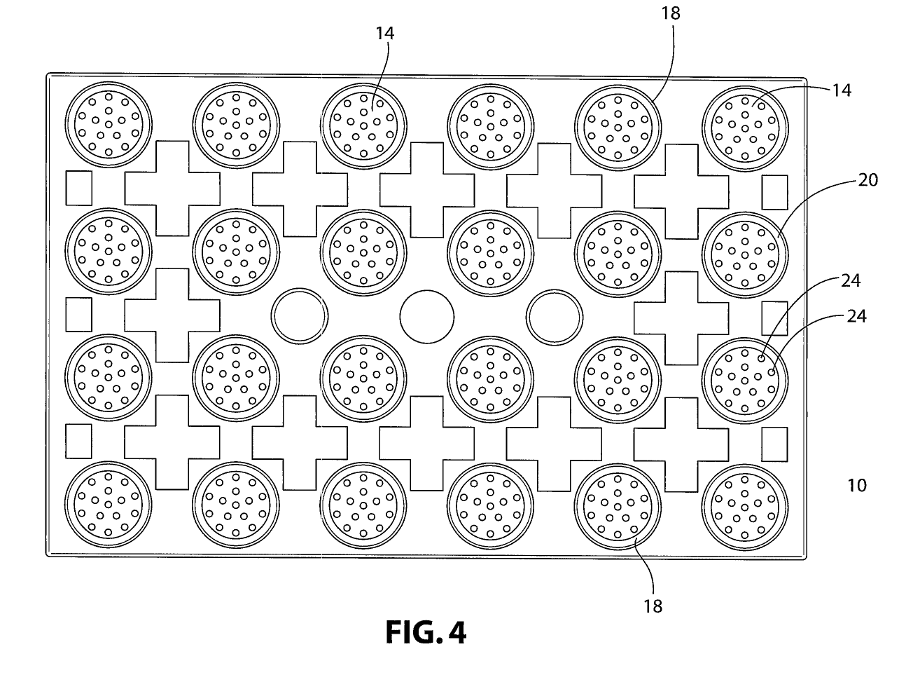

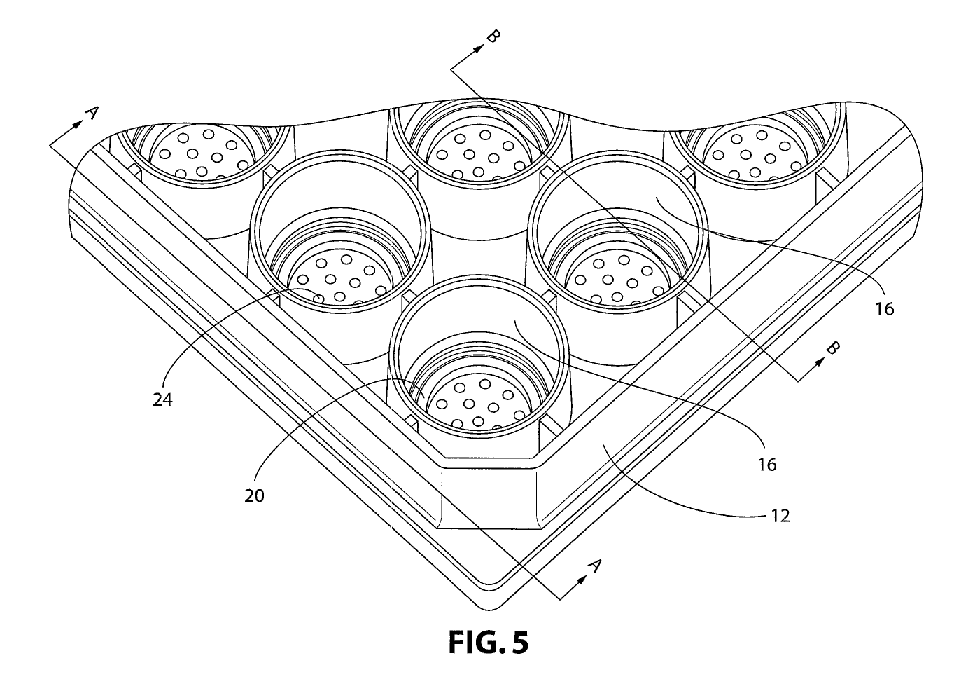

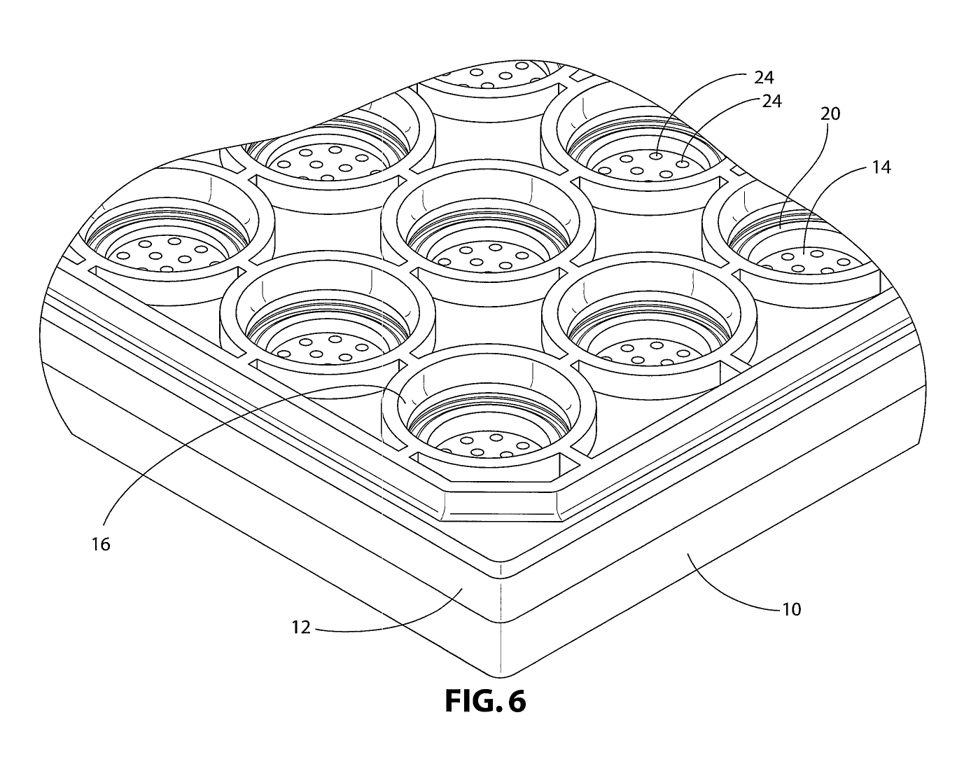

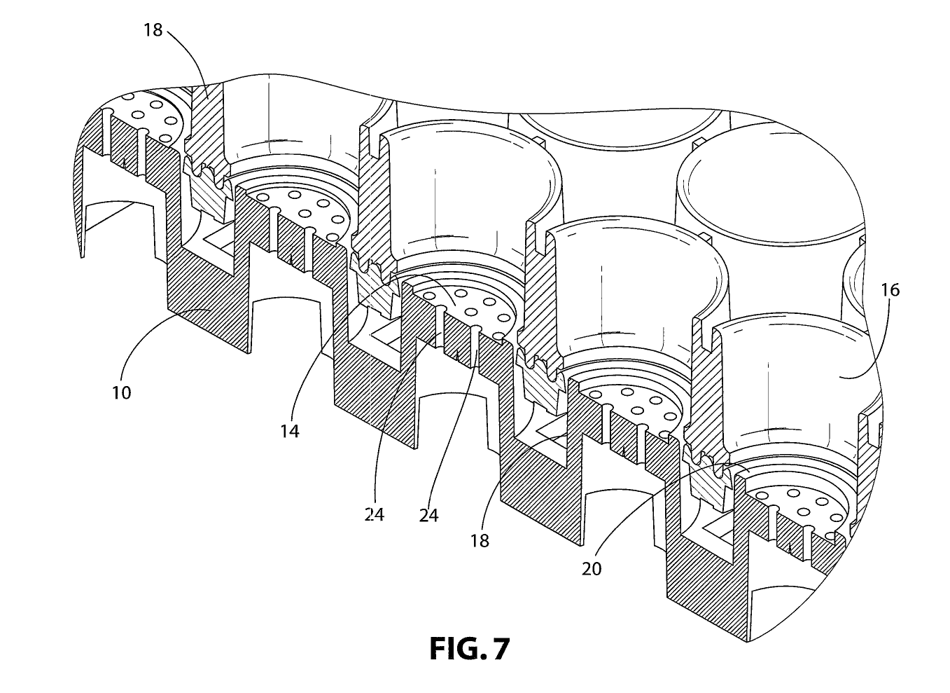

FIGS. 1-4 show several views of a 24-post post plate (10) of an embodiment of the present invention. Whereas, FIGS. 5-7 show the 24-post post plate (10) coupled to a complementary culture plate (12). The post plate (10) is designed to selectively plate cells in two dimensions in the center of each recessed area (14) and/or well (16) of each post (18) of the 24-post post plate (10). Each post (18) has a continuous perimeter (20) that contacts the bottom of a flexible membrane (22) so that the cells will adhere to the flexible membrane (22) positioned over a post (18) of a predetermined diameter. The holes (24) in each post (18) of the post plate (10) are designed to draw the flexible membrane (22) down to conform into the recessed area (14) bounded by the continuous perimeter (20) when vacuum is communicated through the holes (24). The size and placement of the vacuum holes (24) are also important for drawing the flexible membrane (22) down to conform to the recessed area (14) and to result in a uniform strain on the flexible membrane (22). By this method, the flexible membrane (22) forms a cavity into which cells (26) may be deposited to adhere, spread and divide. The height of the continuous perimeter (20) is determined by the desired volume of the region into which the cells (26) are deposited on the flexible membrane (22).

Various continuous perimeter (20) heights for different uses can be designed. For 100 microliters of medium and cells, an optimum wall height is from 500 to 1000 microns. The reason for this wall height is that the flexible membrane (22) is stretched as it is drawn over the continuous perimeter (20) edge. This stretching stiffens the membrane surface and changes the characteristics of the adherent cells. Moreover, excessive strain in any one well (16) affects the strain achieved in the membranes of neighboring wells (16). A wall height between 500 and 1000 microns results in about 2-5% strain in the membrane and has little effect on the adjacent well strains or adherent cells when the vacuum is released bringing the cells to the horizontal plane of the flexible membrane (22).

Reference is now made more particularly to FIGS. 8 and 9 which show human tenocytes stained with crystal violet that were grown for 48 hours on the flexible membrane (22) of a 24 well HTP plate with a flexible membrane growth surface (22). The cells (26) are centered in the middle of the wells (16) so that equibiaxial strain may be applied to each cell culture. The clear region in the periphery has no cells while the central region of the well (16) has a cell sheet (26). If equibiaxial strain were applied from the underside of the flexible membrane (22) using a planar loading post (not show), the membrane would translate across the loading post and only cells located over the loading post would experience strain. Moreover, there would be no cells that would not experience strain or if plated in the peripheral zone, that might experience strain in excess of the intended strain due to hyper stretch in the unbounded annulus of the flexible membrane (22). Other designs of the cell culture device can be implemented to create different cell constructs to fill different needs, several examples are detailed below.

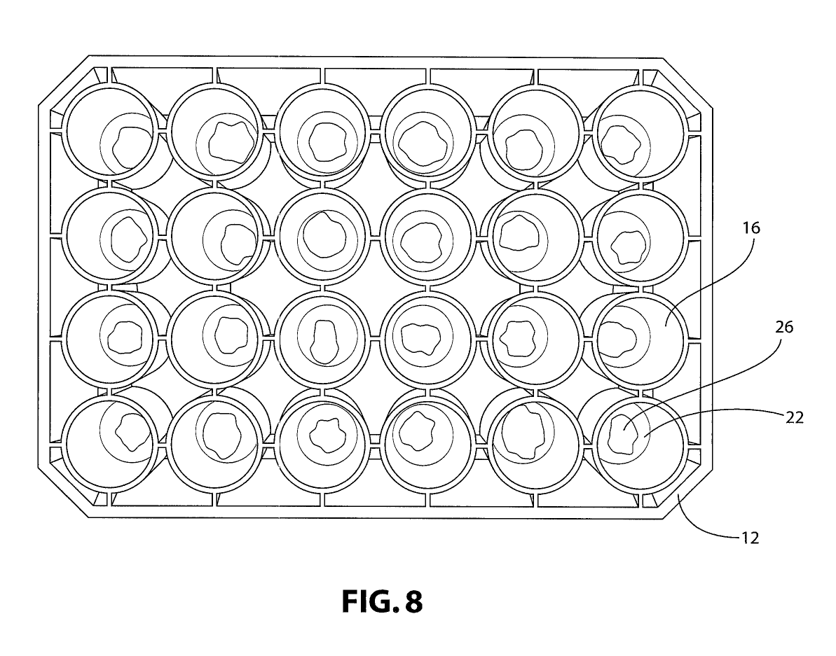

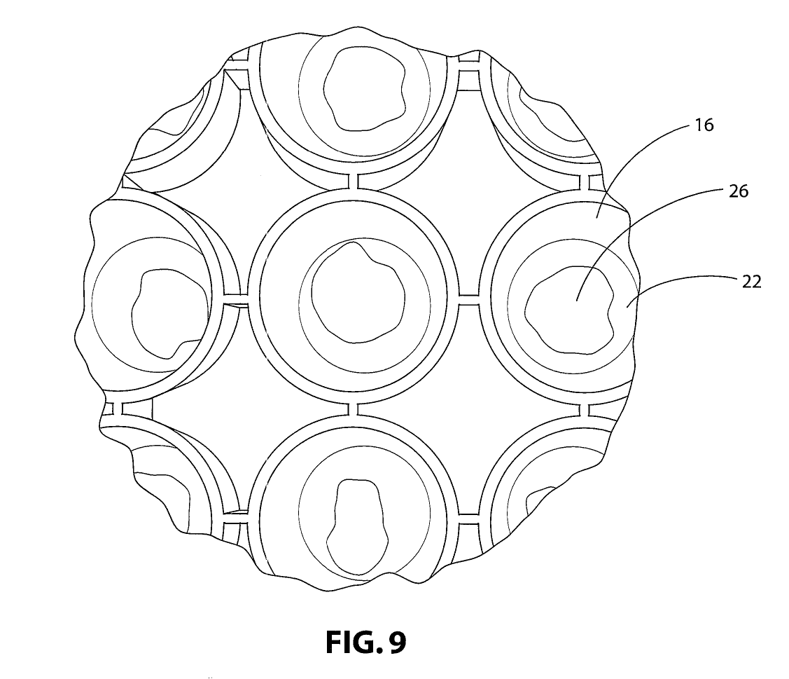

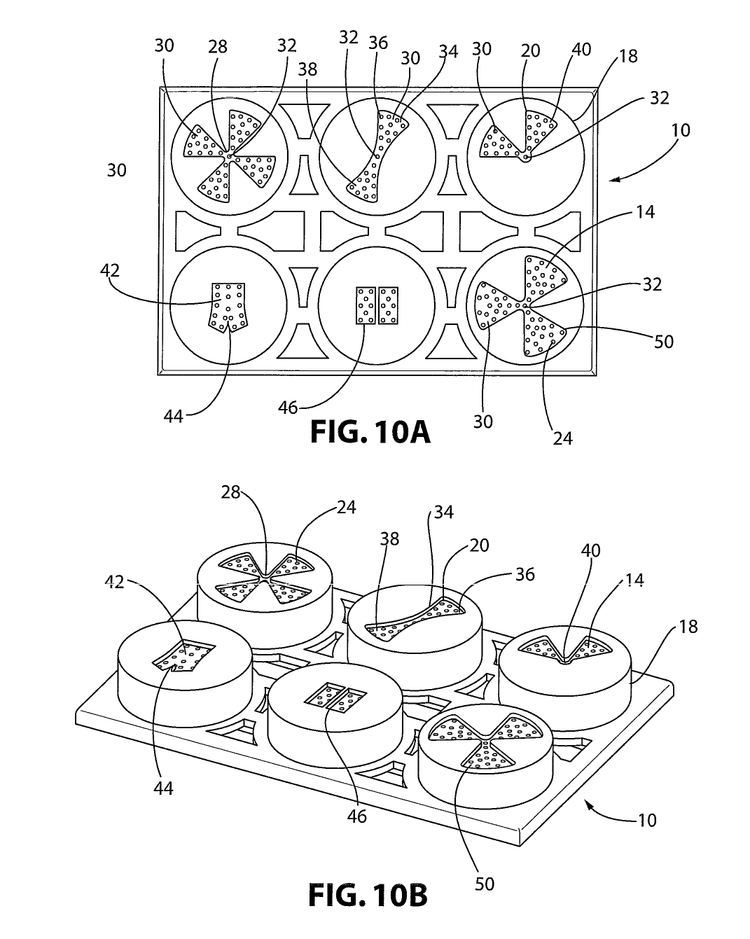

Reference is now made to FIGS. 10A and 10B, which shows a plan view (FIG. 10A) and a perspective view (FIG. 10B) of a 6-post post plate (10) showing six different embodiments of recessed areas (14) designs to create cell constructs in different geometries to test the rates of cell migration toward cells of like or different types, or of ligands potted in a hydrogel in an adjacent trough, or with strain of a given regimen, amplitude, frequency, or duration. The upper left clover leaf design (28), a 4 leaf clover or double propeller design, is such that either 4 different cell types can be plated adjacent to each other in the arms (30) of the cloverleaf design (28) which connect a central portion (32). In addition, should strain be included in a cell migration experiment, then a wedge-shaped loading post (not shown) could be placed beneath the flexible membrane (22) at a prescribed distance from the edges of the cell sheet (26), between each region where cells were plated between any two arms (30) of the clover leaf design (28). In this way, vacuum could be applied at a given level so that the flexible membrane (22) can be drawn downward and thus strain applied to the flexible membrane (22) on which the cells (26) are plated. In the case of the single propeller blade design (34), a wedge-shaped loading post (not shown) would be situated adjacent to the upper propeller blade (36) at the 2:00 position and at 8:00 at the lower propeller blade (38). In the case a cell construct of the upper right design (40), butterfly, a loading post could be applied between the arms (30) or at either side. The lower left design (42) is used to simulate a tear in a tissue, blue jeans design. The crotch or bifurcation (44) of the blue jeans (42) is where a tear can be initiated given that this location can be a strain riser or stress concentrator. In one embodiment, a loading post can be designed to apply strain so that the opposing legs are pulled in opposite directions, simulating a tear that occurs in the supraspinatus tendon. This can be done in 3D by bonding nylon anchors to the membrane on the north and south poles of the construct and depositing cells (26) in a hydrogel between the anchors so that the gel bonds to the tabs connected to the anchors. Then applying force to pull the split ends from each other to simulate the tear.

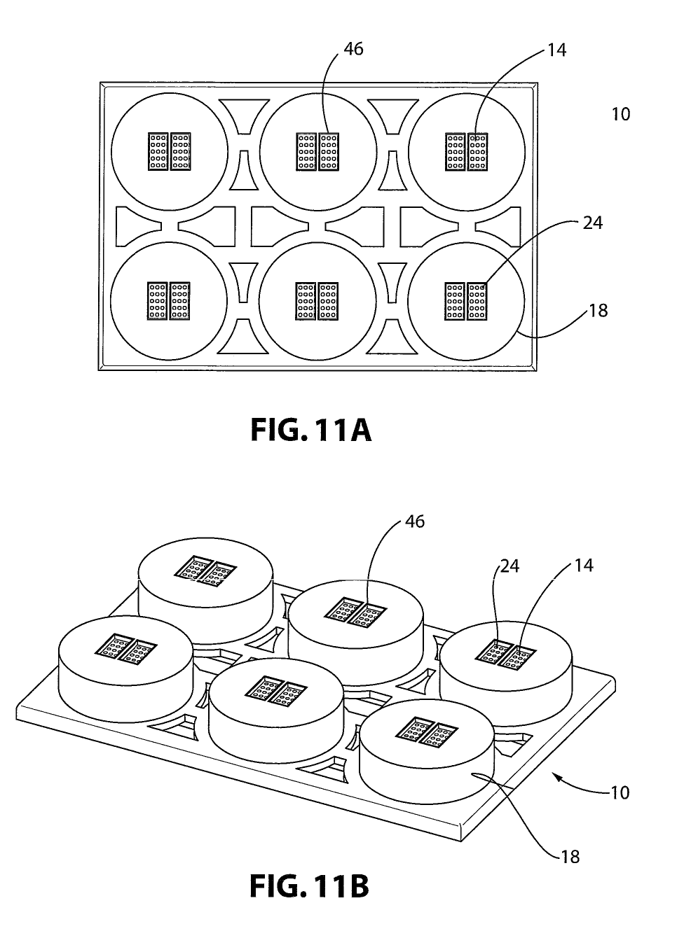

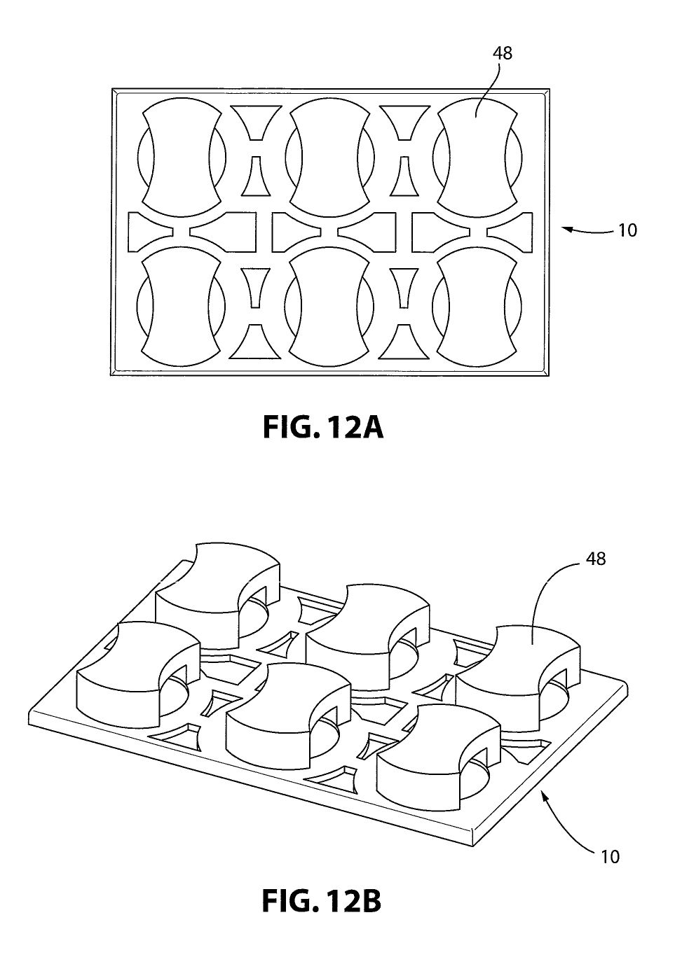

The lower middle design (46) has cells or cells in 3D gel plated in the two rectangular recessed areas (14), further shown in FIGS. 11A-12B. If used in 3D, then the gels will adhere to a nylon tab (not shown) at east and west poles. Strain can be applied uniaxially by a dog-bone shaped loading post (48) placed beneath the cell construct pulling east and west directions and causing separation at the middle of the nylon fabric, causing the cells and gel to tear at that location. The lower right design (50) is similar to upper left and right but can be used to isolate strains.

One can also apply strain at regulated regimens to simulate forces at wound margins. In this way, drugs which affect the mechanisms of cell migration can be tested. Classes of drugs include anti-cancer drugs, cardio-active drugs, drugs that affect molecular processes thought to involve cell motility or the cytoskeleton. The underlying shape-forming devices, are embodied in vacuum-based jigs to draw the flexible membrane (22) down into the desired geometric shape. Once the flexible membrane (22) is drawn to the particular shape, cells can be deposited in the voids, allowed to adhere, then vacuum released to allow the flexible membrane (22) to return to the horizontal plane. Non-adherent cells are washed from the flexible membrane (22) and the shape of the cell area and number of cells in the cell area can be determined. The shape and number of cells can be determined daily until the conclusion of the experiment. One can also deposit multiple cell types or cells in a gel with and without drugs or factors to increase or decrease cell growth or differentiation.

Further to FIG. 10, the post plate (10) can be used in 3D with cells in hydrogels as mentioned above. In this case, the device can be used to simulate a wound or tear in a tissue engineered tissue. Another class of devices includes cell or three dimensional (3D) tissue wounding devices. The concept here is that one can simulate the geometric shape of a tissue in vitro, in a hydrogel model, replete with different cell types that make up the tissue, then wound that tissue in a fashion akin to that which occurs in vivo.

The hydrogel can be a collagen gel, hyaluronic acid, fibrin, or a mixture of the above in a gradient of gel. The matrix from any tissue may be used as a starting source for the gel matrix into which cells may be seeded. The gel matrix may be an acid extract of a connective tissue, such as tendon, ligament, skin, bone, cartilage, or other like tissue. Other materials may be used as the gel matrix, including a collagen gel, a polyglycolic acid, a polylactic acid, agarose, alginate, a silicone gel, or a urethane gel.

If an acid extraction is used, then the acid solution may be neutralized with a base so that the final ionic strength is commensurate with cell survival and matrix polymerization into fibrils where desired. When the gel matrix is an acid extract of a connective tissue, the acid composition is preferably 0.5M acetic acid in water; however, other concentrations and acid formulations may be used. Other extraction solutions may be used including salt solutions. The concentration of the matrix solution can be controlled by a user to form a more loose or compact gel.

For instance, the supraspinatus tendon in the rotator cuff complex is a band of tissue that when torn, has a “V”-shaped tear starting at the proximal end of the tendon in the shoulder. The tendon can be simulated in a geometric shape, such as a rectangular band of hydrogel attached at either long side to a nonwoven mesh. A cell-charged matrix, such as a collagen hydrogel, can be applied between the nylon anchors. One can cast tenocytes from the supraspinatus tendon in a collagen gel connected to a supporting anchor material, such as a nonwoven nylon mesh or like material that is flexible but inelastic. The nylon mesh can be bonded to the underlying Bioflex® flexible bottom silicone elastomer membrane along a few millimeters of one edge. The rest of the nylon mesh anchor is free to move within the confines of the recessed area (14) and/or well (16). The screen or nylon will have a vertical cut from top to bottom, separating the two sides of the nylon. A rectangular recessed area (14) is then placed beneath the rubber membrane to draw the membrane downward to create a void into which to seed cells and collagen hydrogel. Once cast and gelled, the membrane can be released and one will have a “tissue” captured between the two nylon frame edges with cells and gel between. Once the cells compact the matrix, one can apply uniaxial strain from the frame edges on either side and tear the tissue apart, simulating a tear to the supraspinatus tendon.

The present invention can also be used for cell interaction assays. The concept is to fabricate small bore channels as in a microfluidics device, but with vacuum slots, produce a cell seeding pattern, cover the seeded cells with a cover slip or a hydrogel sheet, then score for response on cell pseudopods or connectivity to a second cell population. One skilled in the art would anticipate having a first cell population at the east pole of a chip and a second cell population at the west pole then score for pseudopod growth of one cell toward the other.

The present invention can also be used for cytotoxicity assays. One could use the device of the present invention similarly to the cell interaction, cell migration and wound healing embodiments discussed above and add cells to one bay and a drug to another bay and monitor pseudopod extension or contraction in response to the compound.

The present invention can also be used for cell growth and proliferation assays. For example, by seeding cells in dots in a matrix, then measuring the radial growth of the dividing cells over time as a measure of a cell growth curve.

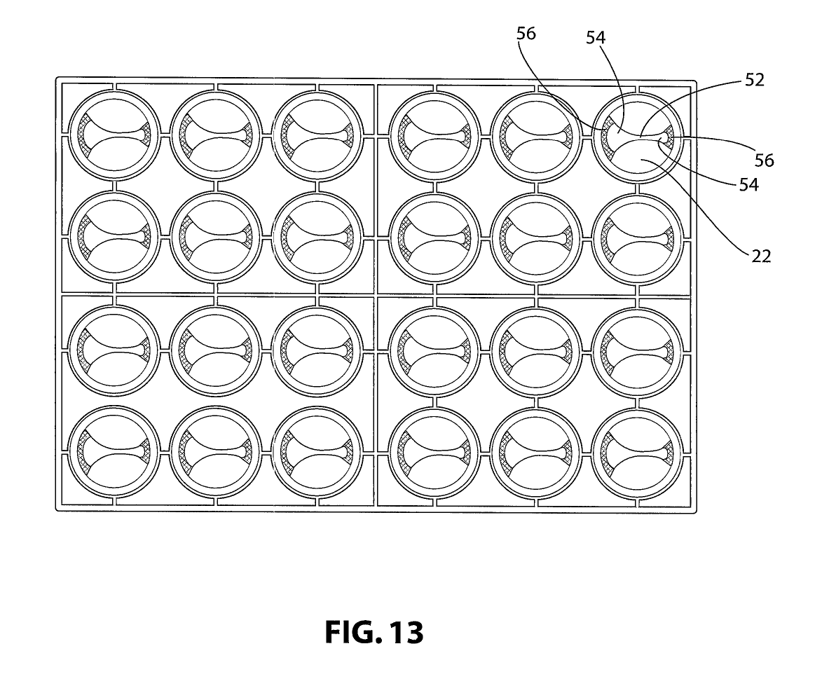

FIG. 13 shows a 24 well HTP plate that has had 24 trapazoidal three dimensional cell-hydrogel constructs (52) created in trapazoidal recessed areas (14) with cell construct ends (54) attached to a nonwoven nylon mesh anchor (56) that is attached to the membrane (22) at anchor ends of the nylon (56) but attached to the cell-hydrogel construct (52) at the cell construct ends (54) at anchor (56) in the nylon. Each construct in this case, has a more broad geometry at the western side of the construct than at the eastern side, to simulate the epimysium attachment to muscle. The narrow end simulates the osseotendinous junction that attaches to bone. This construct simulates the anatomy of an Achilles tendon (i.e., trapazoidal). Moreover, each construct can be subjected to regulated strain by placing a rectangle with curved short ends shaped loading post (48), which is shown in FIGS. 12A and 12B, perpendicularly to the construct, beneath each construct, so that when vacuum is applied, the construct will experience uniaxial strain that is proportional to the level of vacuum applied.

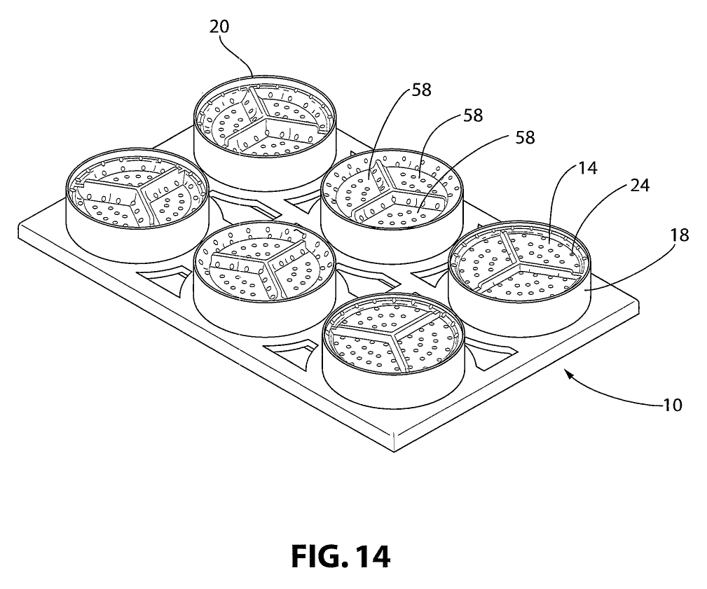

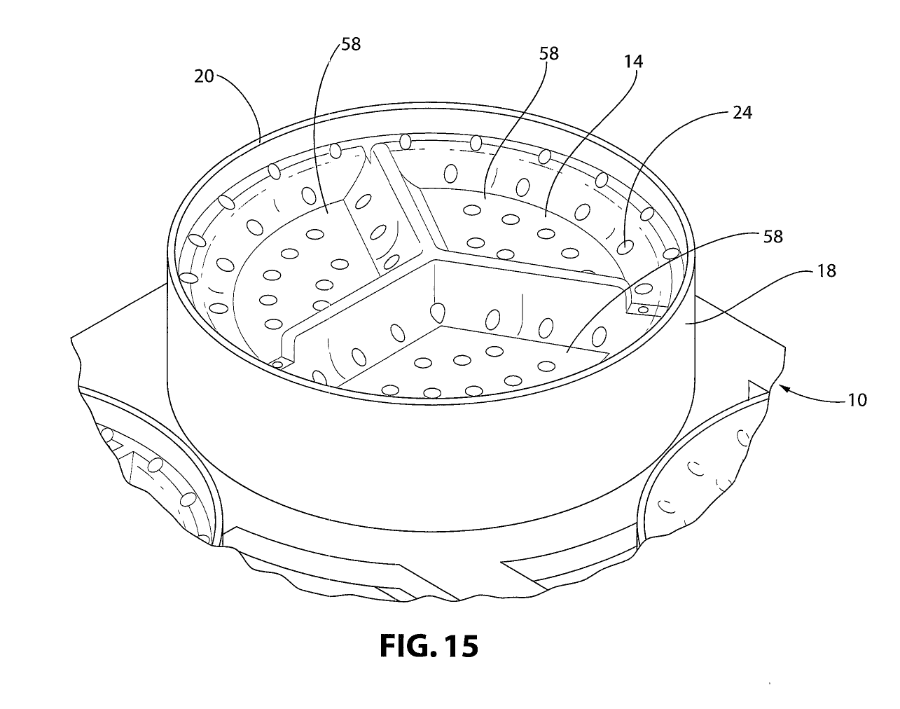

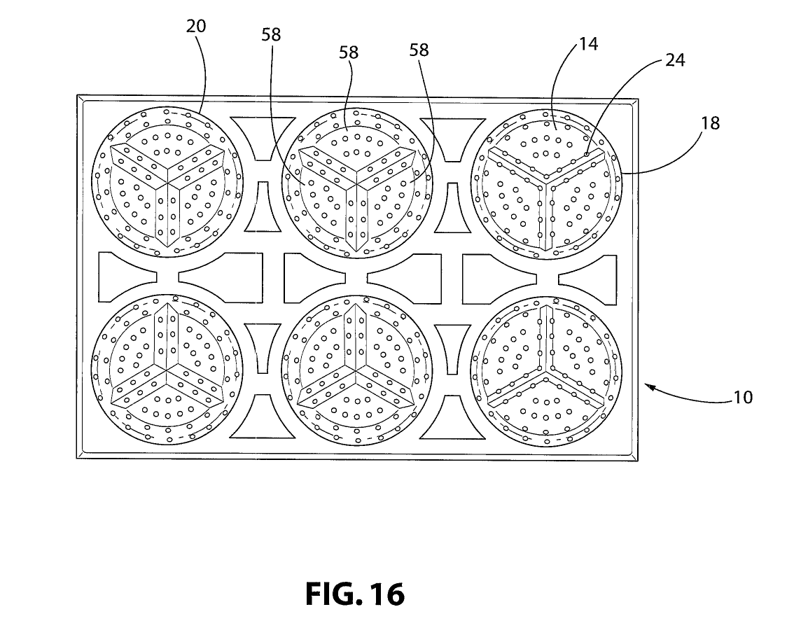

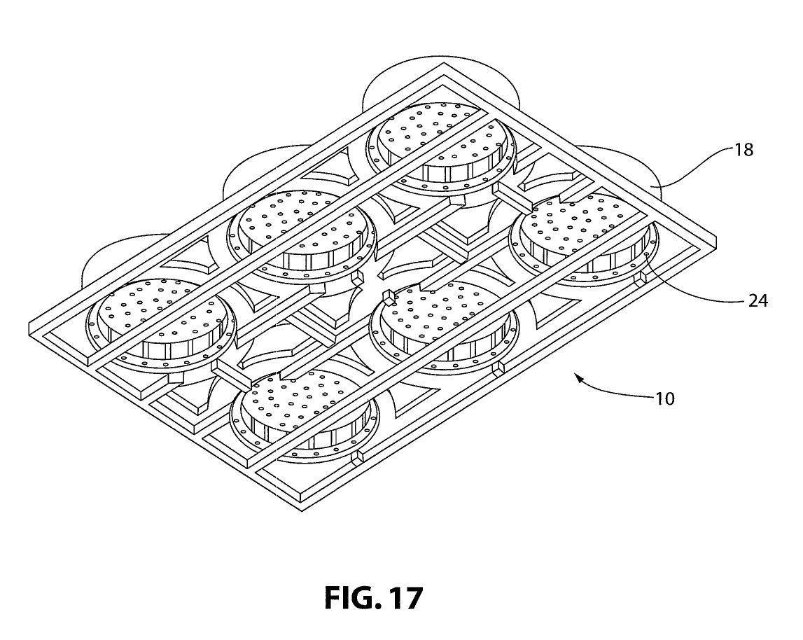

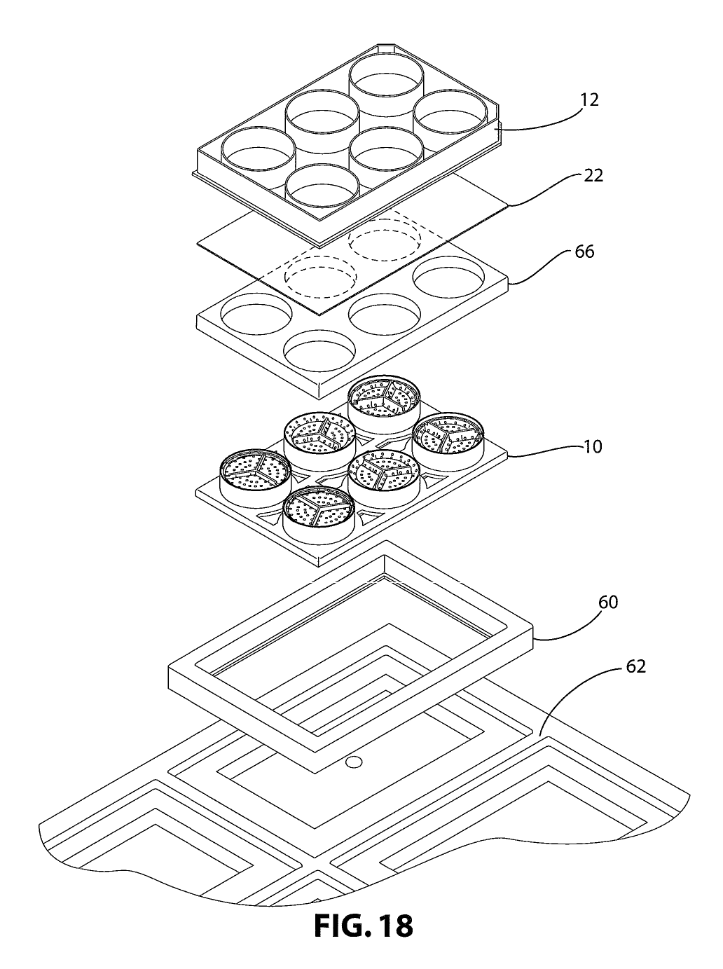

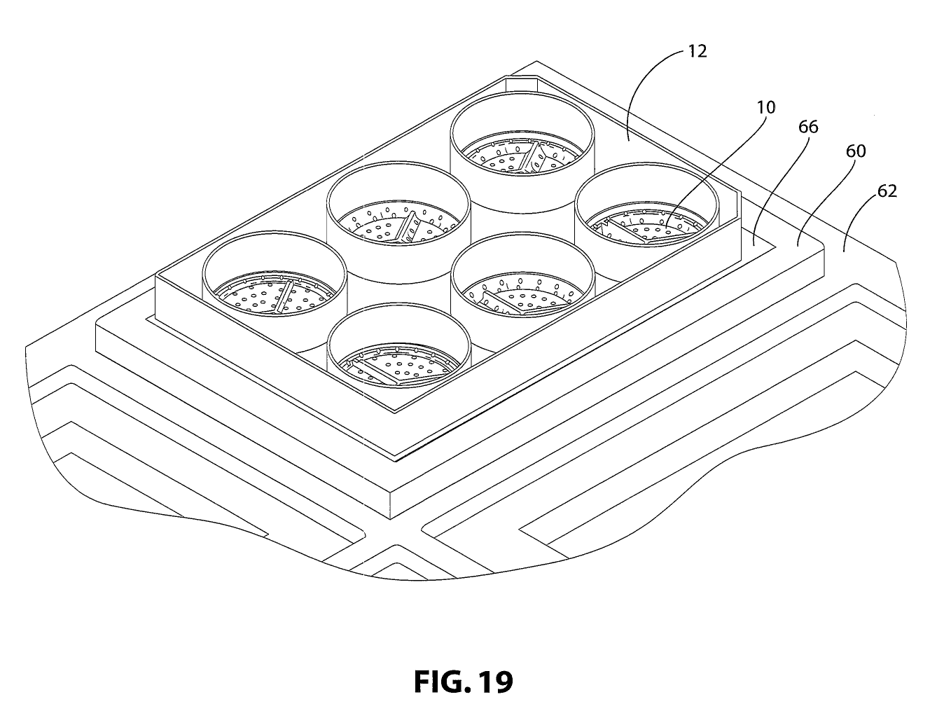

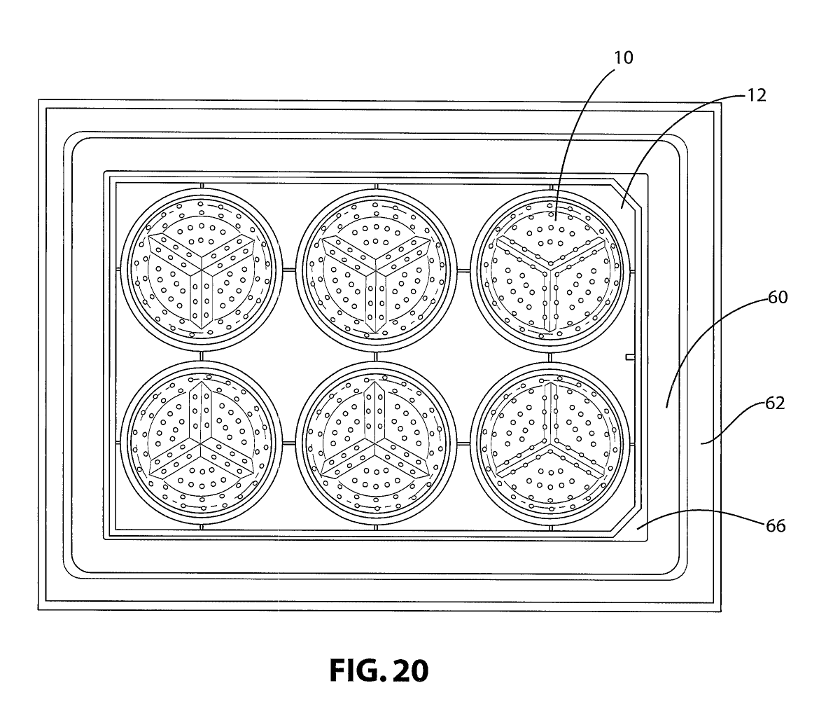

FIGS. 14-20 show an embodiment of the present invention designed to create and simulate the biomechanical, biological, and physiological aspects of the tricuspid heart valve. This heart valve, also known as the right atrioventricular valve, consists of three leaflets (58) that allow the blood to flow in one direction through the heart on the right dorsal side between the right atrium and the right ventricle. This device is used in conjunction with Flexcell's BioFlex® culture plate (12) and a vacuum sealing gasket (60) and baseplate system (62). The vacuum is provided by a vacuum pump (not shown) connected into a baseplate system (62) and through the tricuspid valve post plate (64) to pull down the flexible membrane (22) of the BioFlex® culture plate base (66) via holes (24). This creates three concave leaflets (58) and a suture cuff (68) in the periphery made of a non-woven nylon scaffold, the tricuspid valve post (70) design allows a three dimensional biological leaflet gel to form in the cavities.

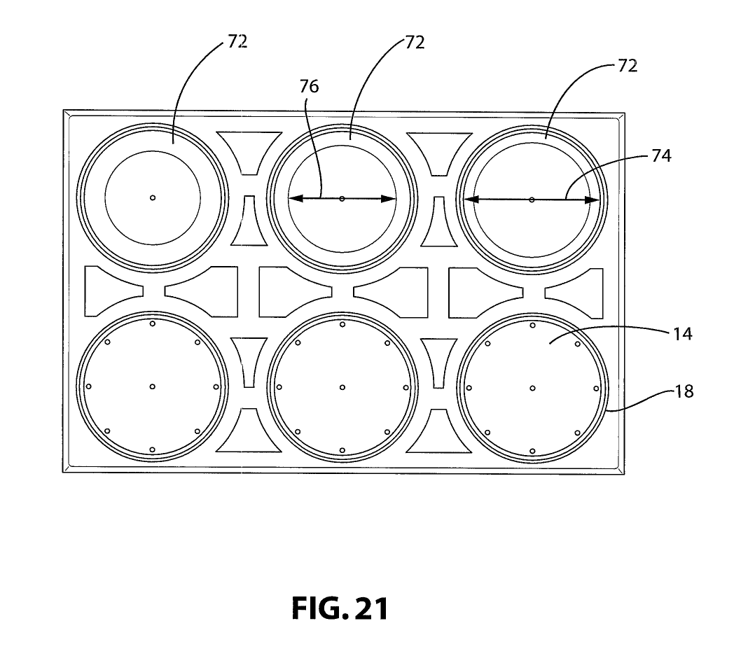

FIGS. 21 and 22 show an embodiment of the present invention including a six-post post plate (10) with a series of rings (72) designed to reduce the recessed area (14) of the posts (18) in order to control the area that cells may be deposited and/or adhered to on the flexible membrane (22), when vacuum is communicated to the flexible membrane (22). The rings (72) can be constructed in various sizes and shapes depending on the desired size and/or shape of the recessed area (14) and depending on the size of the recessed area (14) of the post (18). The outer diameter (74) of each ring (72) determines what size post recessed area (14) the rings (72) can be installed into and the inner diameter (76) of each ring (72) determines the effective recessed area (14) for drawing the flexible membrane (22). Accordingly, the rings (72) can be used to change the shape of the effective recessed area (14) for drawing the flexible membrane (22) in order to pattern cells in various shapes. Referring to FIG. 22, the underside (78) of each ring (72) contains a channel (80) that is configured to communicate with a plurality of holes (24) of the post recessed area (14). The underside (78) of each ring further includes a plurality of slits (82) for connecting the channel (80) to the interior diameter (76) of the ring, which aides in communicating vacuum to the flexible membrane (22).

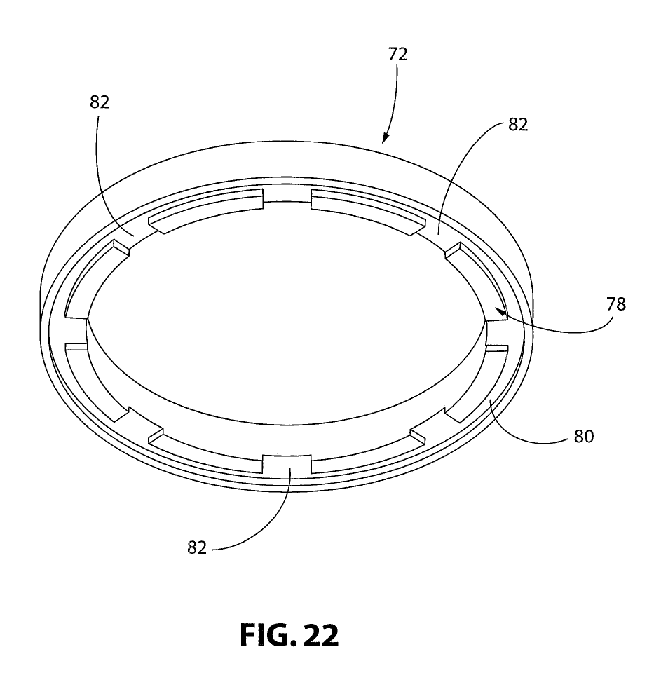

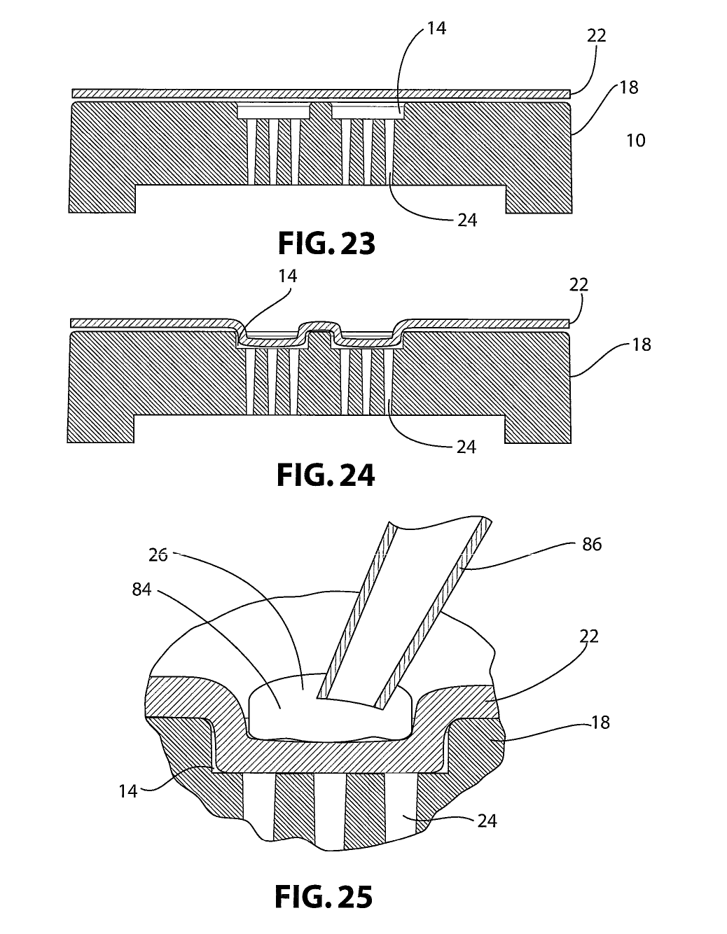

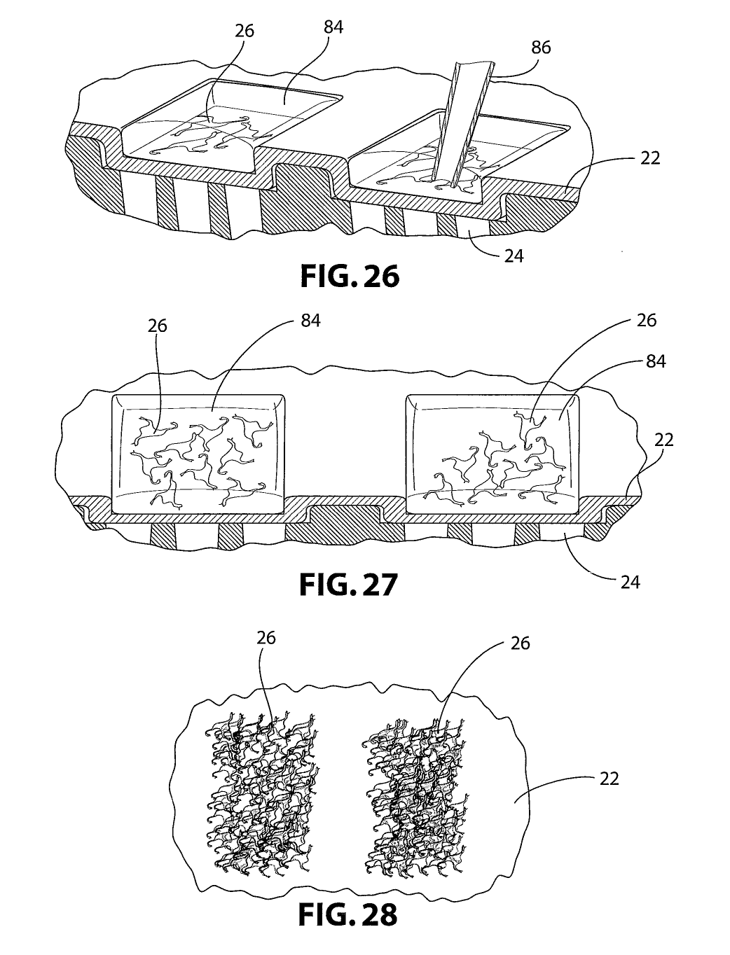

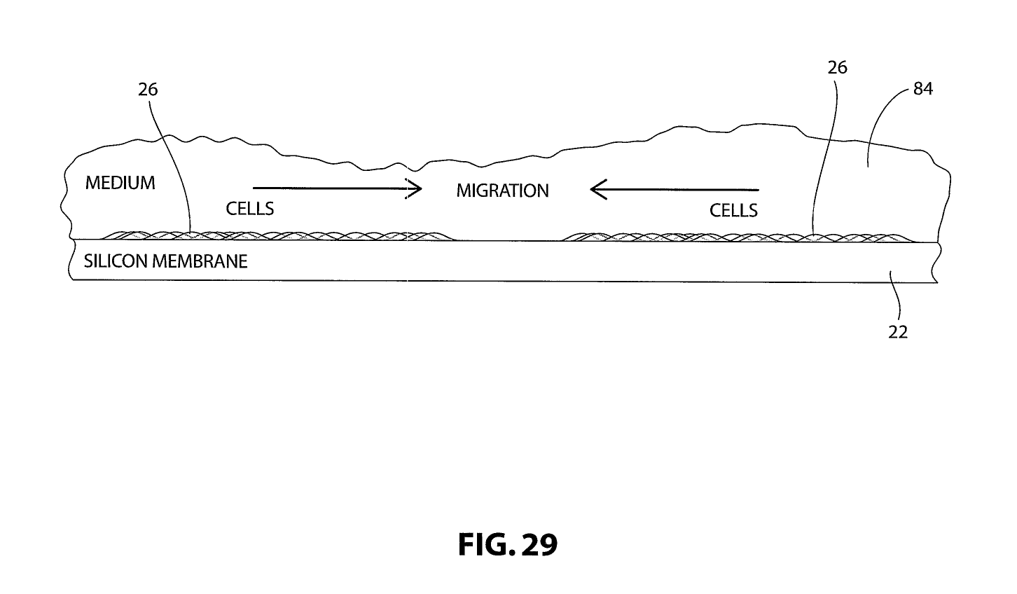

Referring to FIGS. 23-29, a cell migration assay may be performed by using the post station (10) shown in FIGS. 11A and 11B. First, the flexible membrane (22) is positioned over the post (18), as shown in FIG. 23. Then vacuum is communicated to the flexible membrane (22) via holes (24) in the recessed area (14) of the post (18), as shown in FIG. 24. Subsequently, cells (26) with a hydrogel growth medium (84) are deposited on the flexible membrane (22) in the recessed area (14) via a pipette (86), as shown in FIG. 25. After a period of time the hydrogel growth medium (84) is changed via the pipette (86), as shown in FIG. 26. The period of time for changing the growth medium (84) will depend on the type of cells, growth medium composition and other factors of the assay. The cells (26) are then allowed to proliferate during an incubation period until the cells (26) are confluent, as shown in FIG. 27. The vacuum is then released from the flexible membrane (22), as shown in FIG. 28. Additional growth medium and/or hydrogel (84) may be added to the flexible membrane (22) in order to give the cells an environment to migrate. However, in some assays the growth medium and/or hydrogel (84) already on the flexible membrane (22) may be adequate. The cell migration assay now begins and the cells (26) are observed over a period of time to determine the cell migration rate/s of the cells, as shown in FIG. 29. Of course, other aspects of the cells' behavior may be observed using the same method and apparatus.

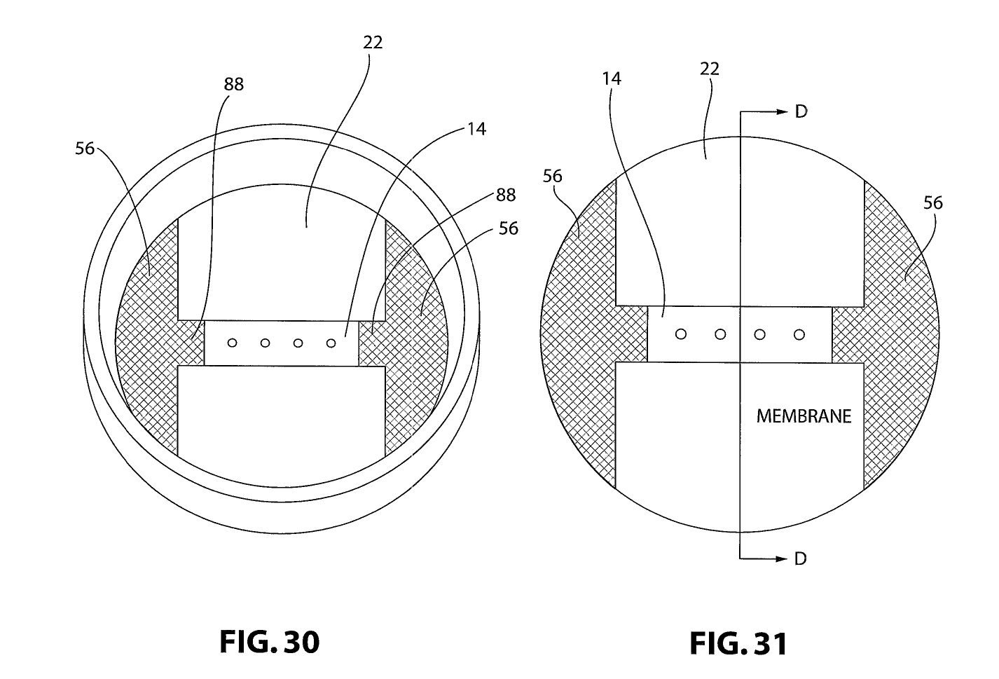

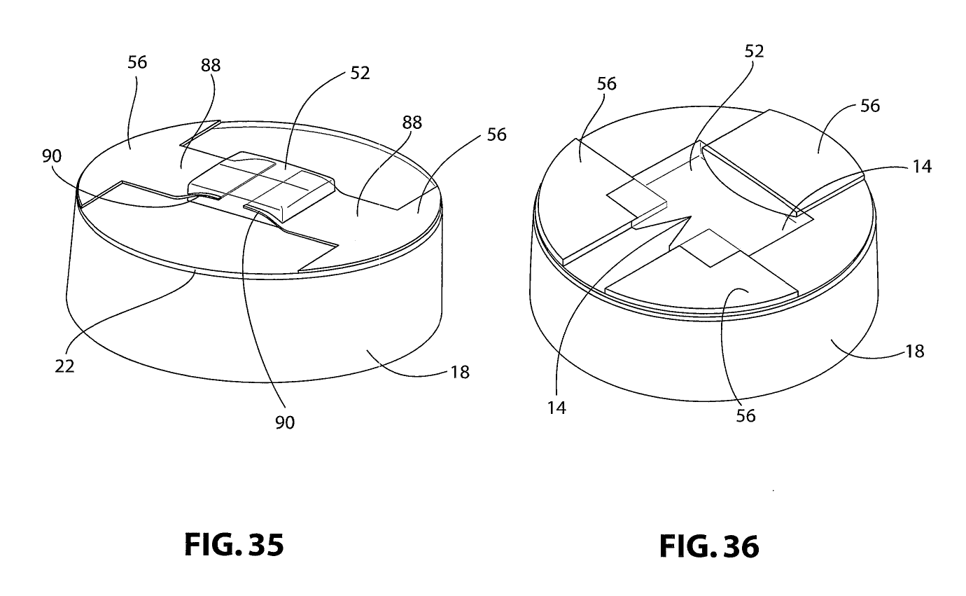

Referring to FIGS. 30-35, an embodiment of a post (18) with a trough shaped recessed area (14) coupled to a well (16) to grow cell cultures according to the present invention is shown.

The anchors (56) are attached at diametrically opposed locations on the top surface of the flexible membrane (22). The anchors (56) may be attached to the flexible membrane (22) using a silicone rubber formulation or other like adhesive or bonding material.

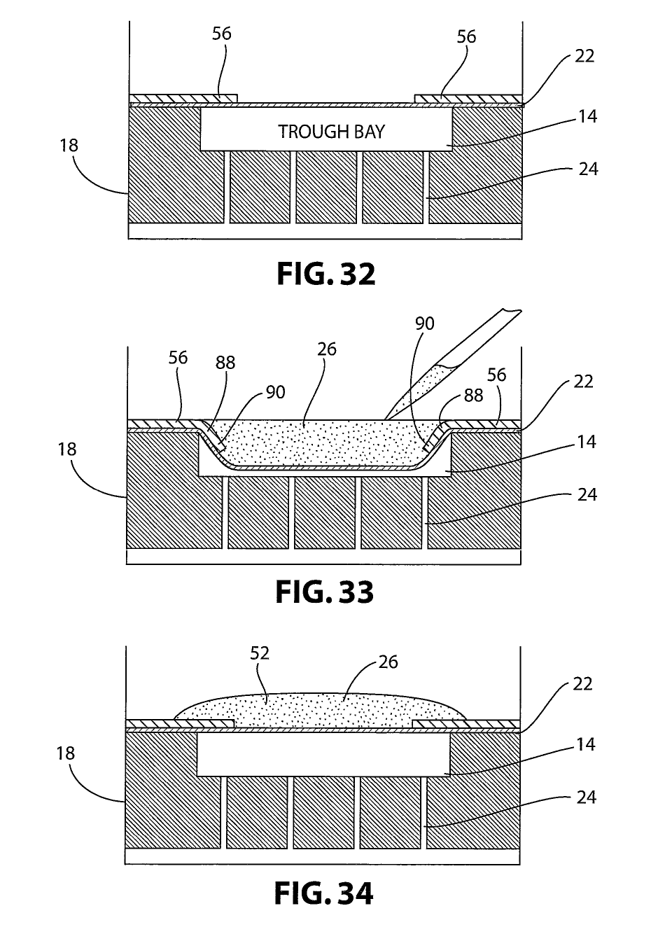

The anchor stem (88) extends from the anchor (56) and is not potted to the flexible membrane (22). Thus, the anchor stem (88) has a free portion (90) to which cells (26) can attach (see FIGS. 32-34) and grow into a cell construct (52). In this configuration, the cells (26) attached to the opposed anchor stems (88) will grow toward each other and, thereafter, intermingle to form a continuous cell construct (52).

The anchor (56) and/or anchor stem (88) may be constructed from materials such as nylon, silk, cotton, polyester, urethane, or other like materials. The material may be solid and/or mesh. The anchor (56) and/or anchor stem (88) may be a layered series of different materials. The anchor stem (88) may be treated with acidic or basic reagents to improve cell attachment. The anchor stem (88) may be treated further with matrix peptides and/or proteins which are absorbed or covalently bonded to the anchor stem (88). The cells (26) may then attach to the anchor stem (88).

The post (18) is positioned to contact the bottom surface of the flexible membrane (22). The post (18) may be cylindrical to fit within the well (16) of the culture plate (12) such that the flexible membrane (22) rests on the continuous perimeter (20) of the post (18).

An elongate recessed area (14) is defined in the post (18) to be adjacent the bottom surface of the flexible membrane (22). Cells alone or in a gel matrix may be supplied to the flexible membrane (22) to form the three-dimensional cell construct (52). Alternatively, a gel matrix alone may be supplied to the flexible membrane (22). When a gel matrix is initially supplied without cells, the gel matrix is allowed to set or at least partially solidify (i.e., polymerize) before the flexible membrane (22) is released from within elongate recessed area (14). Then, cells are supplied to the gel matrix.

The cells are then allowed to grow to form a three-dimensional construct. Cells populating a gel matrix may reorganize the gel matrix and produce their own matrix, thereby adding to the strength of the matrix. In addition to bonding to the matrix, the cells may also bond directly to the anchor (56) and/or anchor stem (88). In this situation, the cells attach and apply force to the anchor (56) and/or anchor stem (88). In so doing, the cells restructure or remodel the gel matrix, resulting in a construct with greater integrity and strength than if only the gel matrix adhered to the anchor stem (88).

Referring to FIG. 36, a cell construct (52) formed in a bifurcated shaped recessed area (14) of a post (18) with three anchors (56) for simulating a tissue tear is shown. The bifurcated cell construct (52) can be created using the above disclosed methods, however with a recessed area (14) relating to the shape of the bifurcated cell construct (52) and with three anchors (56) arranged as shown. This particular arrangement is capable of concentrating strain at the bifurcation (44), which simulates tissue tears when vacuum is communicated to the flexible membrane (22).

It will be understood by those skilled in the art that while the foregoing description sets forth in detail preferred embodiments of the present invention, modifications, additions, and changes might be made thereto without departing from the spirit and scope of the invention.

The invention claimed is:

1. A cell culture apparatus comprising:
a post comprising a recessed area bounded by a continuous perimeter,
a flexible membrane overlaying at least part of the post and overlaying the recessed area, and
a plurality of holes within the recessed area configured to communicate a vacuum to draw said flexible membrane into the recessed area bounded by the continuous perimeter,
wherein the recessed area of the post is of a predetermined shape selected from a group consisting of a rectangular shape, a tricuspid heart valve shape, a bifurcated shape, a cusp shape, an elongate trough shape, and a plurality of arms connected to each other via a connecting portion.

2. The cell culture apparatus of claim 1, further comprising a ring comprising a body having a first end and a second end, and a sidewall extending between the first end and the second end, said sidewall defining a cavity,
said first end of said ring being configured to fit inside the recessed area of the post reducing the length of the continuous perimeter configured to contact the flexible membrane.

3. The cell culture apparatus of claim 2, wherein said first end of said ring defines at least one channel configured to cooperate with said plurality of holes to apply vacuum to draw said flexible membrane to conform into the recessed area bounded by the continuous perimeter of the post.

4. The cell culture apparatus of claim 1, wherein the predetermined shape is the rectangular shape.

5. The cell culture apparatus of claim 1, wherein the predetermined shape is the tricuspid heart valve shape.

6. The cell culture apparatus of claim 1, wherein the predetermined shape is the rectangular shape,
wherein the rectangular shape has a bifurcation at one side of the rectangular shape.

7. The cell culture apparatus of claim 1, wherein the predetermined shape is the bifurcated shape.

8. The cell culture apparatus of claim 1, wherein the predetermined shape is the cusp shape.

9. The cell culture apparatus of claim 1, wherein the predetermined shape is the elongate trough shape.

10. The cell culture apparatus of claim 1, wherein the predetermined shape is the plurality of arms connected to each other via a connecting portion.

11. A cell culture apparatus comprising:
a recessed area bounded by a continuous perimeter,
a flexible membrane overlaying at least part of the recessed area,
a ring comprising a body having a first end and a second end, and a sidewall extending between the first end and the second end, said sidewall defining a cavity,
said second end of said ring being configured to fit inside the recessed area, and
a plurality of holes within the recessed area configured to communicate a vacuum to draw said flexible membrane into the cavity defined by said sidewall when said second end of said ring is inside the recessed area.

12. The cell culture apparatus of claim 11, wherein said second end of said ring defines at least one channel configured to cooperate with said plurality of holes to apply vacuum to draw said flexible membrane to conform into the cavity defined by said sidewall of said ring.

13. The cell culture apparatus of claim 11, wherein the recessed area is of a predetermined shape selected from a group consisting of a circular shape, a rectangular shape, a tricuspid heart valve shape, a bifurcated shape, a cusp shape, an elongate trough shape, and a plurality of arms connected to each other via a connecting portion.

14. The cell culture apparatus of claim 13, wherein the predetermined shape is the circular shape.

15. The cell culture apparatus of claim 13, wherein the predetermined shape is the rectangular shape.

16. The cell culture apparatus of claim 13, wherein the predetermined shape is the tricuspid heart valve shape.

17. The cell culture apparatus of claim 13, wherein the predetermined shape is the rectangular shape,
wherein the rectangular shape has a bifurcation at one side of the rectangular shape.

18. The cell culture apparatus of claim 13, wherein the predetermined shape is the bifurcated shape.

19. The cell culture apparatus of claim 13, wherein the predetermined shape is the cusp shape.

20. The cell culture apparatus of claim 13, wherein the predetermined shape is the elongate trough shape.

21. The cell culture apparatus of claim 13, wherein the predetermined shape is the plurality of arms connected to each other via a connecting portion.

22. The cell culture apparatus of claim 11, wherein the cavity is of a predetermined shape selected from a group consisting of a circular shape, a rectangular shape, a tricuspid heart valve shape, a bifurcated shape, a cusp shape, an elongate trough shape, and a plurality of arms connected to each other via a connecting portion.

23. A method of patterning cells, comprising the steps of:
providing a cell culture apparatus comprising, a flexible membrane over a recessed area having a predetermined shape bounded by a continuous perimeter,
drawing the flexible membrane into the recessed area,
depositing at least one cell and a gel onto the flexible membrane in the recessed area,
allowing the gel to polymerize, and
allowing the cells to proliferate,
wherein the recessed area is of a predetermined shape selected from a group consisting of a rectangular shape, a tricuspid heart valve shape, a bifurcated shape, a cusp shape, an elongate trough shape, and a plurality of arms connected to each other via a connecting portion.

24. The method of patterning cells of claim 23, further comprising the step of:
positioning a flexible cell anchor on the flexible membrane so that at least a portion of the flexible cell anchor is positioned in the recessed area.

25. The method of patterning cells of claim 24, wherein the flexible cell anchor is constructed from a non-woven mesh material.

26. The method of patterning cells of claim 23, wherein the step of drawing the flexible membrane into the recessed area comprises communicating a vacuum to the flexible membrane.

27. The method of patterning cells of claim 23, wherein the predetermined shape is the rectangular shape.

28. The method of patterning cells of claim 23, wherein the predetermined shape is the tricuspid heart valve shape.

29. The method of patterning cells of claim 23, wherein the predetermined shape is the rectangular shape,
wherein the rectangular shape has a bifurcation at one side of the rectangular shape.

30. The method of patterning cells of claim 23, wherein the predetermined shape is the bifurcated shape.

31. The method of patterning cells of claim 23, wherein the predetermined shape is the cusp shape.

32. The method of patterning cells of claim 23, wherein the predetermined shape is the elongate trough shape.

33. The method of patterning cells of claim 23, wherein the predetermined shape is the plurality of arms connected to each other via a connecting portion.
